(19) United States
(12) Patent Application Publication
(10) Pub. No.: US 2024/0032994 A1
(43) Pub. Date: Feb. 1, 2024

(54) ELECTRIC WIRE CONNECTION STRUCTURE, ELECTRIC WIRE CONNECTION METHOD, MEDICAL DEVICE, AND METHOD OF MANUFACTURING MEDICAL DEVICE

(71) Applicant: Proterial, Ltd., Tokyo (JP)

(72) Inventor: Ryuta TAKAHASHI, Tokyo (JP)

(21) Appl. No.: 18/217,116

(22) Filed: Jun. 30, 2023

(30) Foreign Application Priority Data
Jul. 26, 2022 (JP) 2022-118562
Dec. 8, 2022 (JP) 2022-196674

(51) Int. Cl. A61B 18/14; A61B 18/00

(52) U.S. Cl. CPC A61B 18/1492; A61B 2018/00077; A61B 2018/00178; A61B 2018/00083

(57) ABSTRACT

An electric wire connection structure is provided with a plurality of insulated electric wires. Each insulated electric wire includes a core and an outer periphery of a core wire is covered by an insulation coating. The core wire is connected by an electrically conductive bonding material to each of a plurality of pads provided on a substrate at a tip in a longitudinal direction of each of the plurality of insulated electric wires. For each of the plurality of insulated electric wires, an end surface of the core wire and an end surface of the insulation coating are at the same position at the tip in the longitudinal direction, and the electrically conductive bonding material adheres to the end surface of the core wire and the pads.

### CROSS-REFERENCE TO RELATED APPLICATIONS

The present patent application claims the priority of Japanese patent application No. 2022-118562 filed on Jul. 26, 2022, and the priority of Japanese patent application No. 2022-196674 filed on Dec. 8, 2022, and the entire contents thereof are hereby incorporated by reference.

### TECHNICAL FIELD

This invention relates to an electric wire connection structure, an electric wire connection method, a medical device, and a method for manufacturing a medical device.

### BACKGROUND OF THE INVENTION

Conventionally, cables for signal transmission having a plurality of electric wires each with an outer periphery of a core wire being covered by an insulation coating and which together are held in a tubular outer sheath are used in various devices such as, e.g., medical devices. In such cables, the core wire of each of the plurality of electric wires that are led out of the outer sheath at the end of the cable is soldered to a pad on a substrate (i.e., board).

In the cables described in Patent Literatures 1 and 2, the insulation coating of each of the plurality of electric wires drawn out from the outer sheath is removed to expose the core wire over a predetermined length, and the exposed core wire is soldered to a plurality of pads on the surface of the substrate.

### CITATION LIST

Patent Literature 1: JP2011-82042A

Patent Literature 2: JP2000-68007A

### SUMMARY OF THE INVENTION

For example, in catheter cables for medical use that are configured to be inserted into the human body, ultra-thin (i.e., superfine) electric wires are used to reduce the outer diameter of the cable, and the spacing between pads on the substrate is also narrow. Therefore, short circuits are likely to occur between multiple electric wires due to, e.g., the bending of the core wire exposed from the insulation coating, and the connection work between multiple electric wires and the substrate is highly difficult. And this difficulty in connection work is also a factor that increases the cost of equipment using cables.

Therefore, it is an object of the present invention to provide an electric wire connection structure, an electric wire connection method, a medical device, and a method for manufacturing a medical device that enables easy connection of a plurality of electric wires and a substrate.

For solving the above problem, one aspect of the present invention provides: an electric wire connection structure, comprising: a plurality of insulated electric wires each with an outer periphery of a core wire being covered by an insulation coating, the core wire being connected by an electrically conductive bonding material to each of a plurality of pads provided on a substrate at a tip in a longitudinal direction of each of the plurality of insulated electric wires, wherein, for each of the plurality of insulated electric wires, an end surface of the core wire and an end surface of the insulation coating are at a same position at the tip in the longitudinal direction, and the electrically conductive bonding material adheres to the end surface of the core wire and the pads.

For solving the above problem, another aspect of the present invention provides: an electric wire connection method for connecting each of a plurality of insulated electric wires each with an outer periphery of a core wire being covered by an insulation coating to each of a plurality of pads provided on a substrate at a tip in a longitudinal direction of each of the plurality of insulated electric wires, the method comprising: an alignment step of arranging the plurality of insulated electric wires parallel to each other along a predetermined alignment direction; a cutting step of trimming each of the insulated electric wires in such a manner that an end surface of the core wire and an end surface of the insulation coating are at a same position at the tip in the longitudinal direction; and a connecting step of placing the plurality of insulated electric wires on the substrate and connecting the end surface of the core wire to each of the plurality of pads.

For solving the above problem, a still another aspect of the present invention provides: a medical device, comprising: a catheter cable comprising the plurality of insulated electric wires each with the core wire being covered by the insulation coating; and the substrate comprising the plurality of pads to which the core wires of the plurality of insulated electric wires are connected, wherein one of two longitudinal end portions of the catheter cable is configured to be inserted into a human body, wherein the respective core wires of the plurality of insulated electric wires and the plurality of pads of the substrate are connected by the electric wire connection structure as described above.

For solving the above problem, a further aspect of the present invention provides: a method for manufacturing a medical device provided with a catheter cable comprising the plurality of insulated electric wires each with the core wire being covered by the insulation coating, and the substrate comprising the plurality of pads to which the core wires of the plurality of insulated electric wires are connected, wherein one of two longitudinal end portions of the catheter cable is configured to be inserted into a human body, the method comprising: connecting the respective core wires of the plurality of insulated electric wires to the plurality of pads of the substrate by the electric wire connection method as described above.

### Effects of the invention

According to the present invention, it is possible to provide an electric wire connection structure, an electric wire connection method, a medical device, and a method for manufacturing a medical device that enables easy connection of a plurality of electric wires and a substrate.

### BRIEF DESCRIPTION OF DRAWINGS

FIG. 1A is an explanatory diagram of a multi-electrode catheter in use as an example of a medical device according to an embodiment of the present invention.

FIG. 1B is a cross-sectional view of the catheter cable.

FIG. 2A is a plan view showing one side of the substrate to which the plurality of insulated electric wires of the console cable are connected.

FIG. 2B is a plan view showing the other side of the substrate.

FIG. 3A is a plan view showing an electric wire connection body in which a plurality of insulated electric wires and a substrate are combined.

FIG. 3B is an A-sagittal view of FIG. 3A showing the electric wire connection body viewed from the longitudinal direction of the plurality of insulated electric wires.

FIG. 3C is a B-B line cross-sectional view of FIG. 3A.

FIGS. 4A and 4B are explanatory diagrams of the conditions before and after the cutting step.

FIG. 4C and FIG. 4D are explanatory diagrams showing before and after the connecting step.

FIG. 5 is a plan view of the electric wire connection for a comparative example.

FIG. 6A is a plan view showing a modified example in which the positions of the end surfaces of the respective core wires of the plurality of insulated electric wires on the substrate are aligned in a straight line inclined to the longitudinal direction of the plurality of insulated electric wires.

FIG. 6B is a C-C line cross-sectional view of FIG. 6A.

FIG. 7A is a perspective view showing a modified example in which the end surfaces of the respective cores of the plurality of insulated electric wires are slanted inclined surfaces so as to be oriented toward the pad with respect to a direction perpendicular to the substrate.

FIG. 7B is a D-D line cross-sectional view of FIG. 7A.

FIG. 8A is a perspective view showing a modified example in which the end surfaces of the respective cores of the plurality of insulated electric wires are slanted inclined surfaces so as to be oriented toward the opposite side to the pad with respect to a direction perpendicular to the substrate.

FIG. 8B is an E-E line cross-sectional view of FIG. 8A.

FIG. 9A is a configuration diagram of an electric wire connection body according to the second embodiment, viewed from one side of the substrate.

FIG. 9B is a side view of the electric wire connection body according to the second embodiment, viewed from the arrow F direction of FIG. 9A.

FIG. 9C is a configuration diagram of the electric wire connection body according to the second embodiment, viewed from the other side of the substrate.

FIG. 10A is a G-G line cross-sectional view of FIG. 9A.

FIG. 10B is an H-H line cross-sectional view of FIG. 9A.

FIG. 11A is an I-I line cross-sectional view of FIG. 10B.

FIG. 11B is a J-J line cross-sectional view of FIG. 10B.

FIG. 12 is an explanatory diagram of the alignment step and the integration step for the second embodiment.

FIG. 13 is an explanatory diagram showing the removal step for the second embodiment.

FIG. 14 is an explanatory diagram showing the cutting step for the second embodiment.

FIG. 15 is an explanatory diagram showing the connection step for the second embodiment.

FIG. 16A is a configuration diagram of an electric wire connection body according to a modified example of the second embodiment, viewed from one side of the substrate.

FIG. 16B is a K-K line cross-sectional view of FIG. 16A.

FIG. 16C is an L-L line cross-sectional view of FIG. 16A.

### DETAILED DESCRIPTION OF THE INVENTION

The First Embodiment

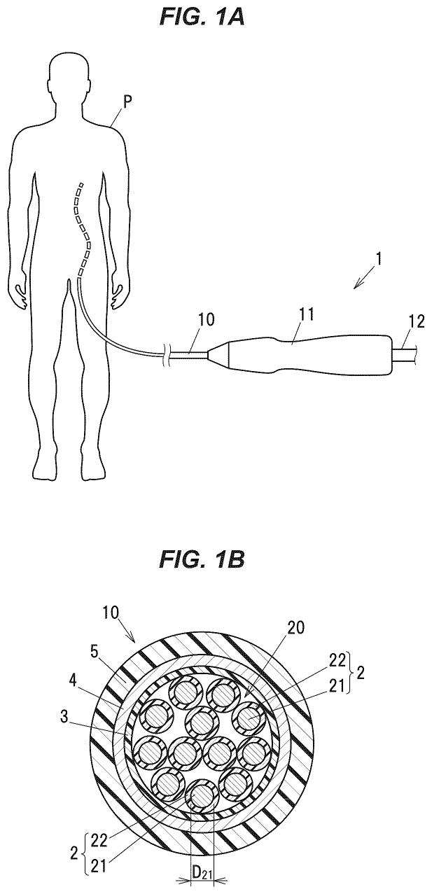

FIG. 1A is an explanatory diagram of a multi-electrode catheter in use as an example of a medical device according to an embodiment of the present invention. FIG. 1B is a cross-sectional view of the catheter cable.

The multi-electrode catheter 1 has a catheter cable 10 and a handle 11 to be operated by an operator such as a physician. One longitudinal end portion of the catheter cable 10 is held within the handle 11, and the other longitudinal end portion is inserted into the human body of the subject P for examination or treatment. In FIG. 1A, the catheter cable 10 is shown as a dashed line in the portion inserted within the human body of the subject P.

As shown in FIG. 1B, the catheter cable 10 has an electric wire bundle 20 composed of a plurality of insulated electric wires (i.e., insulated wires) 2, a binder tape 3 wrapped around the outer periphery of the electric wire bundle 20, a shield conductor 4 disposed around the outer periphery of the binder tape 3 and a tubular jacket 5 as an outer covering disposed around the outer periphery of the shield conductor 4. The jacket 5 is made of fluoropolymer resin, e.g., and includes the electric wire bundle 20, the binder tape 3, and the shield conductor 4. The electric wire bundle 20 is composed of, e.g., five or more insulated electric wires 2 bundled together, and in this embodiment, twelve insulated electric wires 2 are bundled together. Fibrous or string-like fillers (i.e., inclusions) may be placed between the plurality of insulated electric wires 2 in the inner side of the binder tape 3.

Each insulated electric wire 2 has a core wire 21 made of a good electrically conductive metal such as copper and an insulation coating 22 covering the outer periphery of the core wire 21. In the present embodiment, the insulated electric wires 2 are enameled wires, and the outer periphery of the core wire 21, which is made of a solid wire of circular cross-section, is covered with an insulation coating 22 made of a resin composition such as polyurethane. The diameter D₂₁ of the core wire 21 is, e.g., 0.02 mm or more and 0.10 mm or less. In the present embodiment, the core wire 21 corresponds to AWG48 according to the AWG (American Wire Gauge) standard, and the conductor diameter is 0.032 mm (32 μm). The thickness of the insulation coating 22 is, e.g., 0.008 mm (8 μm).

The plurality of insulated electric wires 2 are led out of the jacket 5 in the handle 11. A console cable 12 leads from the handle 11, which connects the handle 11 to an unshown console. The console is an information processing device equipped with a microprocessor, memory, etc., which amplifies signals sent from the human body of the subject P via, e.g., a plurality of insulated electric wires 2, and outputs image signals for displaying the state in the inside of the body of the subject P obtained by the amplified signals.

The handle 11 houses a substrate having a plurality of pads to which the core wire 21 of the plurality of insulated electric wires 2 are connected. Signals sent from the human body of the subject P are relayed by this substrate and sent to the console by the console cable 12. Next, the electric wire connection structure in which the core wires 21 of the plurality of insulated electric wires 2 are connected to the plurality of pads on the substrate will be described.

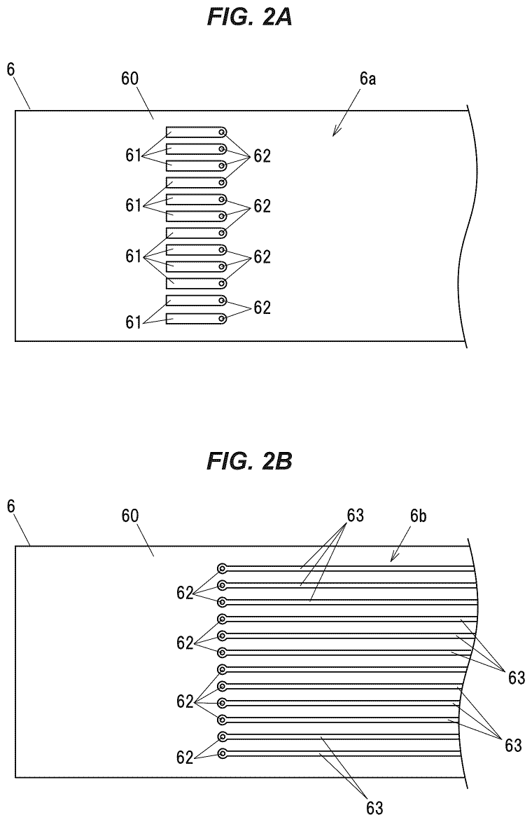

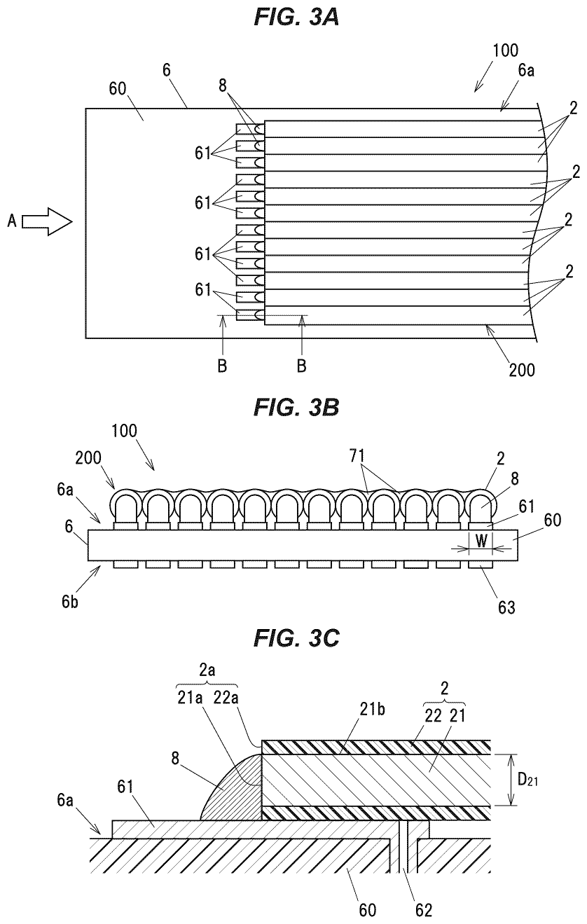

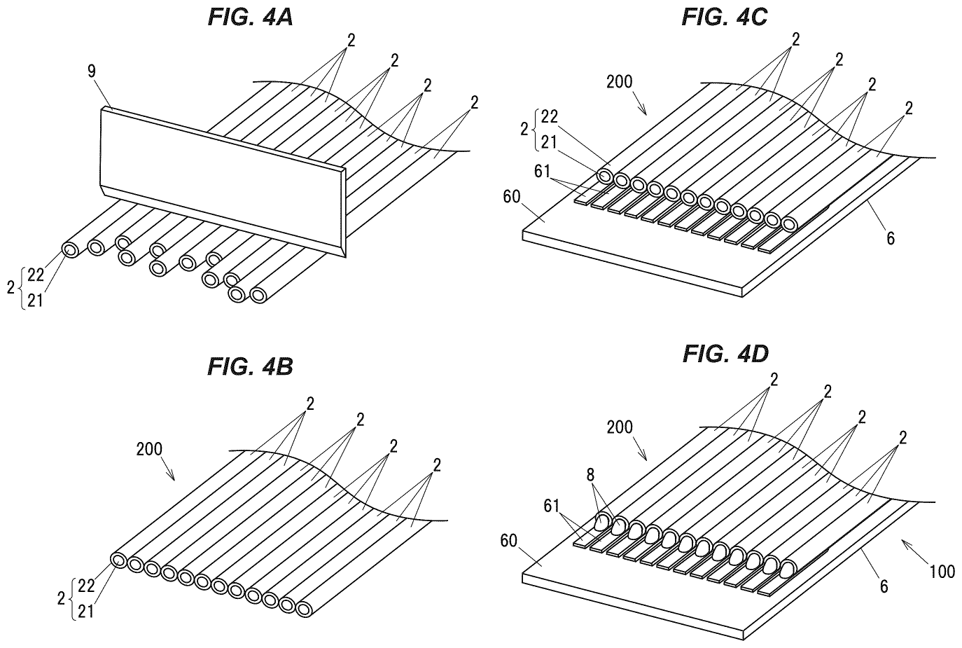

FIG. 2A is a plan view showing one side 6a of the substrate 6 to which the plurality of insulated electric wires 2 of the catheter cable 10 are connected. FIG. 2B is a plan view showing the other side 6b of the substrate 6. FIG. 3A is a plan view showing an electric wire connection 100 in which a plurality of insulated electric wires 2 and a substrate 6 are combined. FIG. 3B is an A-sagittal view of FIG. 3A showing the electric wire connection 100 viewed from the longitudinal direction of the plurality of insulated electric wires 2. FIG. 3C is a B-B line cross-sectional view of FIG. 3A.

The plurality of insulated electric wires 2 are integrated on the substrate 6 to form a strip-shaped multi-core wire array 200. The term “on the substrate 6” here refers to a position in close proximity to and facing the substrate 6. In this embodiment, as shown in FIG. 3B, the plurality of insulated electric wires 2 are integrated by bonding the insulation coatings 22 of the plurality of insulated electric wires 2 together with an adhesive 71. In FIG. 3A and FIGS. 4A to 7B below, the adhesive 71 is omitted.

The plurality of insulated electric wires 2 have the core wire 21 cut at the same position as the insulation coating 22 at the tip of each longitudinal direction, and the end surface 21a of the core wire 21 and the end surface 22a of the insulation coating 22 are at the same position in the longitudinal direction of the insulated electric wires 2 as shown in FIG. 3C.

The plurality of insulated electric wires 2 are arranged parallel to each other on the substrate 6, and when the plurality of insulated electric wires 2 are viewed from a direction perpendicular to one side 6a of the substrate 6 as shown in FIG. 3A, the end surfaces 21a of the respective core wires 21 are aligned in a straight line. In this embodiment, the positions of the end surfaces 21a of the core wires 21 of the plurality of insulated electric wires 2 on the substrate 6 are aligned in a straight line perpendicular to the longitudinal direction of the plurality of insulated electric wires 2.

The substrate 6 is an FPC (flexible printed circuit substrate), and a plurality of pads 61 are provided on the surface of a flat substrate member 60. The pads 61 are electrodes to which the core wires 21 are connected. The substrate member 60 is made of an insulating material, such as polyimide, and is flexible. The substrate 6 is provided with a through-hole 62 that penetrates the substrate member 60 in the thickness direction and a linear wiring pattern 63. A plurality of pads 61 are connected to the respective core wires 21 of a plurality of insulated electric wires 2. The substrate 6 may be a solid substrate that is not flexible.

The pads 61 and the wiring patterns 63 are metal foils formed by etching, and their thickness is, e.g., 1 μm or more and 16 μm or less. The core wire 21 of the insulated electric wire 2 is electrically connected to the terminals of an unshown electronic component mounted on the substrate 6, e.g., via the pad 61, the through-hole 62, and the wiring pattern 63.

In the examples shown in FIGS. 2A and 2B, a plurality of pads 61 are provided only on one side 6a of the substrate 6, and these pads 61 are connected to wiring patterns 63 provided on the other side 6b of the substrate 6 by the through-holes 62. A plurality of pads 61 may also be provided on the other side 6b of the substrate 6, and a plurality of core wires 21 of insulated electric wires 2 may be connected to these pads 61.

In the example shown in FIG. 2A, the pads 61 are formed in a rectangular shape long in the longitudinal direction of the plurality of insulated electric wires 2. However, the pads 61 need only be formed at least at the portion where the core wires 21 are connected. The shape of pad 61 is not limited to a rectangular shape, but may be square, circular, or oval, for example. The width W of the pads 61 in a direction perpendicular to the longitudinal direction of the plurality of insulated electric wires 2 is equivalent to the diameter D₂₁ of the core wire 21, and a predetermined interval is provided between adjacent pads 61.

As shown in FIG. 3C, each of the plurality of insulated electric wires 2 has an end surface 21a of the core wire 21 exposed from the insulation coating 22 at the tip in the longitudinal direction, which is connected to the pad 61 by solder 8 as an electrically conductive bonding material. The solder 8 adheres to the end surface 21a of the core wire 21 and the connection surface 61a of the pad 61, electrically connecting and securing the core wire 21 to the pad 61. The electrically conductive bonding material is not limited to solder, but may be, e.g., electrically conductive adhesive.

In this embodiment, the entire circumference of the peripheral surface 21b of the core wire 21 at the longitudinal tip of each of the plurality of insulated electric wires 2 is covered with an insulation coating 22. The tip surface 2a of the insulated electric wires 2 is one continuous plane with the end surface 22a of the insulation coating 22 and the end surface 21a of the core wire 21. In this embodiment, the end surface 21a of the core wire 21 and the end surface 22a of the insulation coating 22 are perpendicular to the longitudinal direction of the insulated electric wire 2 and to one side 6a of the substrate 6. In this embodiment, at the tip surface 2a of the insulated electric wire 2, the insulation coatings 22 of the adjacent insulated electric wires 2 are in contact with each other.

Next, an electric wire connection method for connecting the core wire 21 of each of the plurality of insulated electric wires 2 to the pad 61 provided on the substrate 6 will be described. This electric wire connection method comprises the following steps: an alignment step in which a plurality of insulated electric wires 2 are aligned parallel to each other along a predetermined alignment direction, an integration step in which the plurality of insulated electric wires 2 aligned in the alignment step are integrated, a cutting step in which the tips of the plurality of insulated electric wires 2 are trimmed, and a connection step in which the plurality of insulated electric wires 2 are placed on the substrate 6 and the core wires 21 are connected to the plurality of pads 61 are connected to each of the plurality of pads. The multi-electrode catheter 1 is manufactured using this electric wire connection method.

FIGS. 4A and B are explanatory diagrams showing the conditions before and after the cutting step. In the cutting step, the plurality of insulated electric wires 2 are trimmed so that the end surface 21a of the core wire 21 and the end surface 22a of the insulation coating 22 are at the same position at the respective longitudinal tips. As a result, the end surface 21a of the core wire 21 is exposed at the respective longitudinal tips of the plurality of insulated electric wires 2, and the peripheral surface 21b of the core wire 21 is covered by the insulation coating 22. In this embodiment, the plurality of insulated electric wires 2 are integrated by the integration step to form a multi-core wire array 200, and the ends of the plurality of insulated electric wires 2 are cut in a batch by the blade tool 91. The blade tool 91 cuts both the core wire 21 and the insulation coating 22 of each of the plurality of insulated electric wires 2. The plurality of insulated electric wires 2 are cut at the same position in the longitudinal direction of the core wire 21 and the insulation coating 22 at their respective ends.

FIGS. 4C and 4D are explanatory diagrams showing the conditions before and after the connecting step. In the connection step, the multi-core wire array 200 is fixed to the substrate 6, for example by bonding, and the end surface 21a of the core wire 21 is connected to the pad 61 by the solder 8. The specific method of this soldering is not particularly limited, but it may be done, e.g., by heating and melting the cream solder applied to the pad 61, or by heating and melting a spherical solder ball placed on the pad 61, or by pick-soldering to the end surface 21a of the core wire 21 of the insulated electric wire 2, or by spraying molten solder. The end surface 21a of the core wire 21 may be connected to the pad 61 by electrically conductive adhesive instead of the solder 8.

Comparative Example

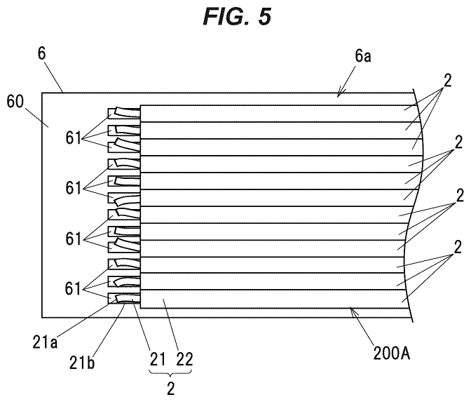

FIG. 5 is a plan view of a multi-core wire array 200A for a comparative example together with a substrate 6. In this multi-core wire array 200A, insulated electric wires 2 having a core wire 21 and an insulation coating 22 are integrated parallel to each other as in the multi-core wire array 200 of the above embodiment, but the insulation coating 22 is removed over a predetermined length at the tip of the insulated electric wire 2 to expose the peripheral surface 21b of the core wire 21.

In this multi-core wire array 200A, the bending and tilting of the core wire 21 in the portion where the insulation coating 22 has been removed cause the core wire 21 of adjacent insulated electric wires 2 to easily short circuit each other when soldered. In particular, when the diameter of the core wire 21 is 0.1 mm or less, the core wire 21 itself has low rigidity and is prone to bending, and the spacing between the pads 61 is narrow, so short circuits due to solder bridges and the like frequently occur during work in the connection step.

Effects of the First Embodiment

According to the first embodiment of the invention described above, the peripheral surface 21b of the core wire 21 at the tip of the plurality of insulated electric wires 2 is covered by the insulation coating 22 and only the end surface 21a of the core wire 21 is exposed, and this end surface 21a is connected to the pad 61 on the substrate 6, thus preventing the occurrence of short circuits during the connection step. This makes it possible to easily connect the insulated electric wires 2 to the substrate 6 while suppressing the occurrence of short circuits during the connection step. Also, as in the comparative example above, it is not necessary to remove the insulation coating 22 over a predetermined length, and working of the insulated electric wires 2 is also facilitated. Furthermore, since the plurality of insulated electric wires 2 are integrated, the ends of the plurality of insulated electric wires 2 can be cut and trimmed together in a batch in the cutting step.

Modified Example of the First Embodiment

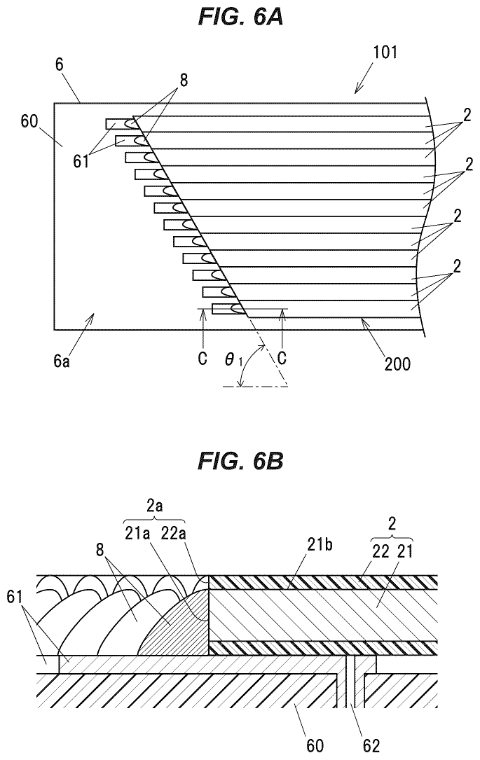

Next, electric wire connectors 101-103 pertaining to modified examples of the first embodiment will be described with reference to FIGS. 6A to 8B. In these modified examples, the shape of the tip of the plurality of insulated electric wires 2 cut in the cutting step is different from that of the above embodiments. In FIGS. 6A to 8B, components corresponding to those described in the first embodiment above are marked with the same characters as those in FIGS. 2A to 5, and redundant explanations are omitted.

FIG. 6A is a plan view showing a modified example in which the positions of the end surfaces 21a of the core wire 21 of each of the plurality of insulated electric wires 2 on the substrate 6 are aligned in a straight line inclined to the longitudinal direction of the plurality of insulated electric wires 2. FIG. 6B is a C-C line cross-sectional view of FIG. 6A.

As shown in FIG. 6A, when the plurality of insulated electric wires 2 are viewed from a direction perpendicular to one side 6a of the substrate 6, the respective tip surfaces 2a of the plurality of insulated electric wires 2 are aligned along a straight line inclined at an angle θ₁ to the longitudinal direction of the plurality of insulated electric wires 2. The angle θ₁ is smaller than 90 degrees, e.g., 30 degrees or more and 70 degrees or less.

In the electric wire connection method for manufacturing the electric wire connection 101 for this modified example, in the cutting step, one end of the plurality of insulated electric wires 2 is cut so that the positions of the end surfaces 21a of the core wire 21 of each of the plurality of insulated electric wires 2 on the substrate 6 are aligned along a straight line inclined with respect to the longitudinal direction and alignment direction of the plurality of insulated electric wires 2.

According to this modified example, in addition to the effects of the above embodiment, the distance between the end surfaces 21a of the core wire 21 of the adjacent insulated electric wires 2 becomes longer, and the connection work between the core wire 21 and the pad 61 becomes easier.

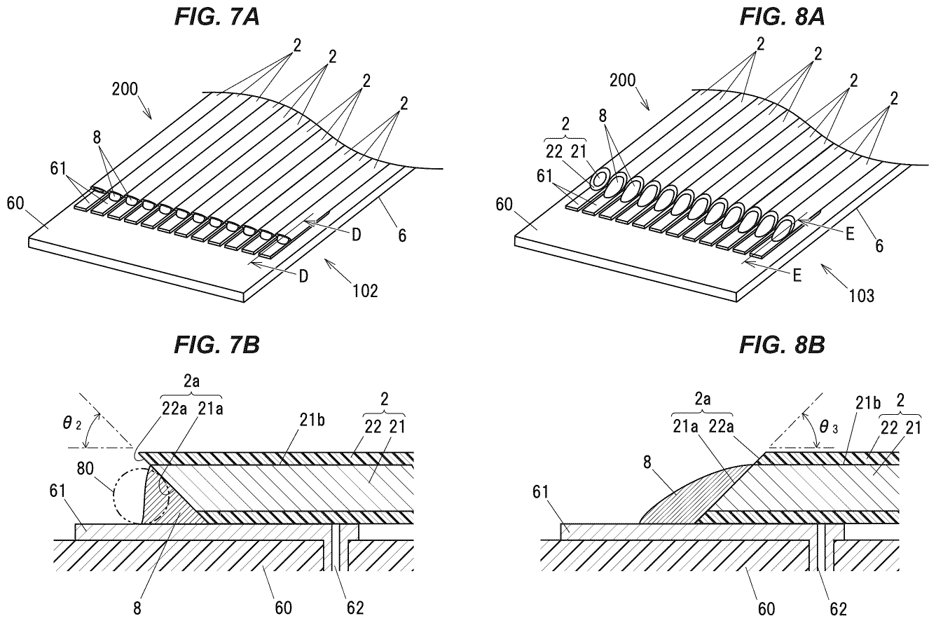

FIG. 7A is a perspective view of a modified example in which the end surface 21a of the core wire 21 of each of the plurality of insulated electric wires 2 is an inclined plane slanted with respect to the direction perpendicular to the substrate 6, and this inclined end surface 21a is oriented toward the pad 61. FIG. 7B is a D-D line cross-sectional view of FIG. 7A. In FIG. 7A, the connection between the core wire 21 and the pad 61 by solder 8 is not yet completed for one insulated electric wire 2 of the plurality of insulated electric wires 2.

As shown in FIG. 7B, the end surface 21a of the core wire 21 of each of the plurality of insulated electric wires 2 is inclined with respect to the longitudinal direction of the plurality of insulated electric wires 2. The inclination angle θ₂ of the end surfaces 21a with respect to the longitudinal direction of the insulated electric wires 2 is, e.g., 30 degrees or more and 60 degrees or less.

In the electric wire connection method for manufacturing the electric wire connection 102 according to this modified example, in the cutting step, the end surface 21a of the core wire 21 of each of the plurality of insulated electric wires 2 is an inclined surface inclined with respect to the longitudinal direction of the plurality of insulated electric wires 2. In the connection step, the plurality of insulated electric wires 2 are placed on the substrate 6 so that the end surface 21a of the core wire 21 is oriented toward the pad 61, and the end surface 21a of the core wire 21 is connected to the pad 61 by solder 8. In this step, as shown by the double-dotted line in FIG. 7B, a spherical solder ball 80 is placed between the end surface 21a of the core wire 21 and the pad 61, and this solder ball 80 is heated and melted to perform soldering. In the cutting step, the end surface 21a of the core wire 21 may be cut at an angle to the longitudinal direction of each of the plurality of insulated electric wires 2 to make the end surface 21a of the core wire 21 an inclined surface, or the plurality of insulated electric wires 2 may be cut perpendicular to the longitudinal direction and then the core wire 21 may be shaved to make the end surface 21a an inclined surface.

According to this modified example, in addition to the effects of the above embodiments, the area of the end surface 21a of the core wire 21 becomes wider, which makes the work of connecting the core wire 21 to the pad 61 easier.

FIG. 8A is a perspective view of a modified example in which the end surface 21a of the core wire 21 of each of the plurality of insulated electric wires 2 is an inclined plane inclined with respect to a direction perpendicular to the substrate 6, and this inclined end surface 21a is oriented toward the opposite side to the pad 61. FIG. 8B is an E-E line cross-sectional view of FIG. 8A. In FIG. 8A, the connection between the core wire 21 and the pad 61 by solder 8 is not yet completed for one insulated electric wire 2 of the plurality of insulated electric wires 2.

As shown in FIG. 8B, the end surface 21a of the core wire 21 of each of the plurality of insulated electric wires 2 is inclined with respect to the longitudinal direction of the plurality of insulated electric wires 2. The inclination angle θ₃ of the end surfaces 21a with respect to the longitudinal direction of the insulated electric wires 2 is, e.g., 30 degrees or more and 60 degrees or less.

In the electric wire connection method for manufacturing the electric wire connection 103 according to this modified example, in the cutting step, the end surface 21a of the core wire 21 of each of the plurality of insulated electric wires 2 is an inclined surface inclined with respect to the longitudinal direction of the plurality of insulated electric wires 2. In the connection step, the plurality of insulated electric wires 2 are placed on the substrate 6 so that the end surface 21a of the core wire 21 is oriented toward the opposite side of the pad 61, and the end surface 21a of the core wire 21 is connected to the pad 61.

According to this modified example, in addition to the effects of the above embodiment, the area of the end surface 21a of the core wire 21 becomes wider, which makes the connection work between the core wire 21 and the pad 61 easier. In addition, since the end surface 21a of the core wire 21 points to the opposite side of the pad 61, it is easier to check the state of the connection between the core wire 21 and the pad 61 after the connection step.

The Second Embodiment

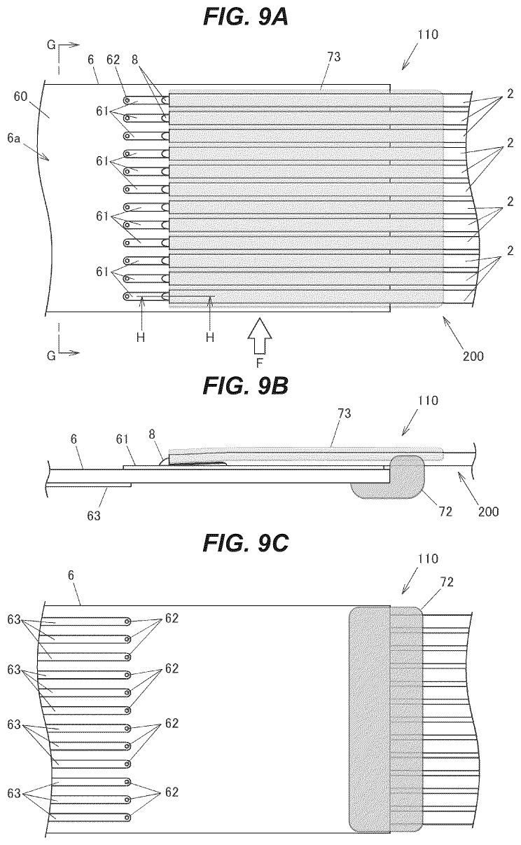

Next, the second embodiment of the invention will be described with reference to FIGS. 9A to 15. In FIGS. 9A to 15, components corresponding to those described in the first embodiment above are marked with the same characters as those in FIGS. 2A to 5, and redundant explanations are omitted.

Similar to the electric wire connection structure of the first embodiment, the electric wire connection structure of this embodiment consists of a plurality of insulated electric wires 2, each of which has a core wire 21 connected to a plurality of pads 61 provided on a substrate 6 by means of solder 8 as an electrically conductive bonding material at the respective longitudinal tips of the electric wires, but in this embodiment, a plurality of insulated electric wires 2 are spaced apart from each other and arranged in parallel on the substrate 6. In this embodiment, a circumferential portion of the insulation coating 22 at the tip of each of the plurality of insulated electric wires 2 is removed to expose the peripheral surface 21b of the core wire 21, and solder 8 as an electrically conductive bonding material is adhered to this exposed peripheral surface 21b of the core wire 21.

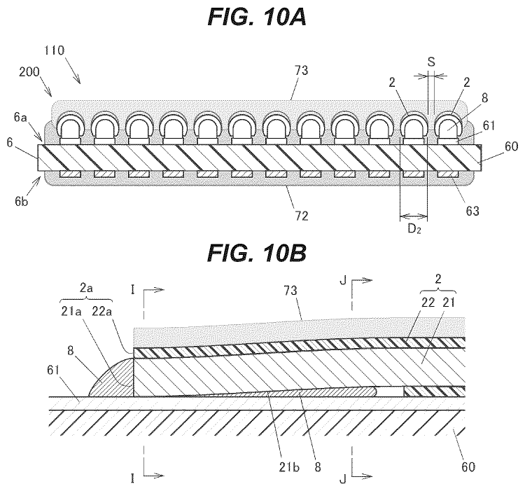

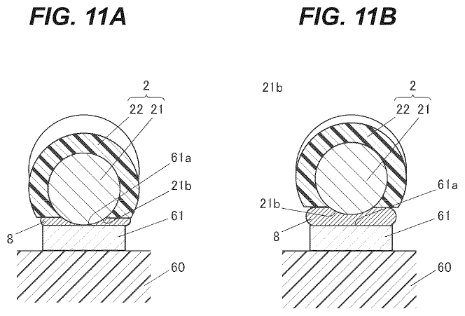

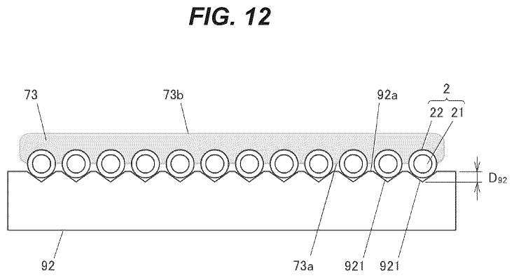

FIGS. 9A to 9C show the electric wire connection 110. FIG. 9A is a configuration diagram thereof, viewed from one side 6a of the substrate 6, FIG. 9B is a side view thereof, viewed from the arrow F direction, and FIG. 9C is a configuration diagram thereof, viewed from the other side 6b of the substrate 6. FIG. 10A is a G-G line cross-sectional view of FIG. 9A, and FIG. 10B is a H-H line cross-sectional view of FIG. 9A. FIG. 11A is an I-I line cross-sectional view of FIG. 10B, and FIG. 11B is a J-J line cross-sectional view of FIG. 10B. FIGS. 12 to 15 are explanatory diagrams of the electric wire connection method.

The electric wire connection 110 is used for a multi-electrode catheter 1, similar to the electric wire connection 100 of the first embodiment. The plurality of insulated electric wires 2 have a core wire 21 and an insulation coating 22 cut at the same position in the longitudinal direction at their respective tips, and the end surface 21a of the core wire 21 and the end surface 22a of the insulation coating 22 are at the same position in the longitudinal direction of the insulated electric wire 2.

The plurality of insulated electric wires 2 are fixed to the substrate 6 by the adhesive 72. In FIGS. 9A to 10B, the adhesive 72 is shown in dark gray. The adhesive 72 adheres to the other side 6b at the edge of the substrate 6 and to a portion of the plurality of insulated electric wires 2 near the edge of the substrate 6. The adhesive 72 may be adhered to the one side 6a of the substrate 6.

In this embodiment, the plurality of insulated electric wires 2 are fixed and integrated at predetermined intervals in the alignment direction by the electric wire fixing member 73. In FIGS. 9A to 15, the electric wire fixing members 73 are shown in light gray. In FIGS. 11A and 11B, the electric wire fixing member 73 is omitted. The electric wire fixing member 73 is made of resin, for example.

In this embodiment, the plurality of insulated electric wires 2 are fixed with a predetermined spacing in the alignment direction, which widens the spacing between the pads 61 and more reliably prevents short circuits between the pads 61. Even if the diameter of each of the plurality of insulated electric wires 2 is formed larger than the standard dimension within the tolerance range, the position of the core wire 21 of each of the plurality of insulated electric wires 2 can be aligned with the position of the pad 61.

The spacing S (see FIG. 10A) between the insulated electric wires 2 in the alignment direction of the plurality of insulated electric wires 2 integrated by the electric wire fixing member 73 is smaller than the diameter D₂ of the insulated electric wires 2. The desirable range of the spacing S relative to the diameter D₂ of the insulated electric wires 2 is 7% or more and 25% or less. If the ratio of the spacing S to the diameter D₂ of the insulated electric wires 2 is less than 7%, it is difficult to obtain the above effect due to the spacing of the plurality of insulated electric wires 2, and if this ratio exceeds 25%, it will result in the substrate 6 becoming larger.

In the present embodiment, a circumferential portion of the insulation coating 22 at the tip of each of the plurality of insulated electric wires 2 is removed to expose the peripheral surface 21b of the core wire 21, and the solder 8 is adhered to this exposed peripheral surface 21b of the core wire 21. The insulation coating 22 has the part of the core wire 21 corresponding to the pad 61 side removed. The adhesion of solder 8 to the peripheral surface 21b of the core wire 21 in addition to the end surface 21a of the core wire 21 in a part of the circumferential direction of the core wire 21 enhances the fixing strength of the core wire 21 to the pad 61.

Furthermore, in this embodiment, the core wire 21 at the tip of each of the plurality of insulated electric wires 2 is bent so as to approach the pad 61. The amount by which the core wire 21 approaches the pad 61 due to this bending is an amount corresponding to the thickness of the insulation coating 22. The core wire 21 is bent in such a way that the distance between the end surface 21a and the pad 61 is shortened and the core wire 21 can be more securely joined to the pad 61.

Next, the electric wire connection method of this embodiment will be described. As in the first embodiment, the electric wire connecting method of this embodiment comprises an alignment step in which a plurality of insulated electric wires 2 are aligned parallel to each other along a predetermined alignment direction, an integration step in which a plurality of insulated electric wires 2 aligned in the alignment step are integrated together, a cutting step in which the tips of multiple insulated electric wires 2 are trimmed so that the end surface 21a of the core wire 21 and the end surface 22a of the insulation coating 22 are aligned at the same position in the longitudinal direction, and a connecting step in which the plurality of insulated electric wires 2 are placed on the substrate 6 and the core wire 21 is connected to each of the plurality of pads 61, and further, has a removing step in which the circumferential part of the insulation coating 22 on the tip of the plurality of insulated electric wires 2 is removed to expose the peripheral surface 21b of the core wire. These steps will be described more specifically below.

FIG. 12 are explanatory diagrams showing specific examples of the alignment and integration steps. In the alignment step, an alignment jig 92 having a plurality of holding grooves 921 extending parallel to each other that hold a portion of each of the plurality of insulated electric wires 2 is used to hold each of the plurality of insulated electric wires 2 in the plurality of holding grooves 921, thereby spacing the plurality of insulated electric wires 2 in parallel with each other. The groove depth D₉₂ of the holding groove 921 from the plurality of insulated electric wires 2-side face 92a in the alignment jig 92 is shallower than the diameter D₂of the insulated electric wires 2, and a portion of the insulated electric wires 2 protrudes from the holding groove 921.

In the integration step, with the plurality of insulated electric wires 2 held in the plurality of holding grooves 921 of the alignment jig 92, the liquid electric wire fixing member 73 before solidification is adhered to the opposite side to the alignment jig 92 in the plurality of insulated electric wires 2 and solidified. The electric wire fixing member 73 should be heat resistant to the extent that it is not affected by the heat generated in the insulated electric wires 2 by the electric current flowing in the core wire 21. The electric wire fixing member 73 should also be viscous to the extent that it does not adhere to the surface 92a on the plurality of insulated electric wires 2-side of the alignment jig 92 in the integration step. For example, a cyanoacrylate based adhesive can be suitably used as the electric wire fixing member 73.

For convenience of description, the surface on the alignment jig 92 side in the electric wire fixing member 73 is hereinafter referred to as the lower surface 73a, and the opposite side is referred to as the upper surface 73b. The electric wire fixing member 73 does not adhere to the alignment jig 92, and a gap (space) is formed between the lower surface 73a of the electric wire fixing member 73 and the surface 92a on the plurality of insulated electric wires 2-side of the alignment jig 92. A part of each of the plurality of insulated electric wires 2 protrudes from the lower surface 73a of the electric wire fixing member 73 and a gap (space) is also formed between this protruding part in the alignment direction of the plurality of insulated electric wires 2.

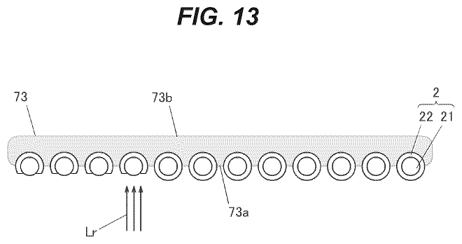

FIG. 13 shows a specific example of the removal step. In the removal step, the insulation coating 22 on the plurality of insulated electric wires 2 in the portion protruding from the lower surface 73a of the electric wire fixing member 73 is removed over a predetermined length in the longitudinal direction of the insulated electric wires 2. FIG. 13 shows an example of removing the insulation coating 22 by irradiating laser light Lr. However, the method of removing the insulation coating 22 is not limited to laser irradiation. The peripheral surface 21b of the core wire 21 exposed in the portion where the insulation coating 22 has been removed faces the pad 61 in the connection step described below.

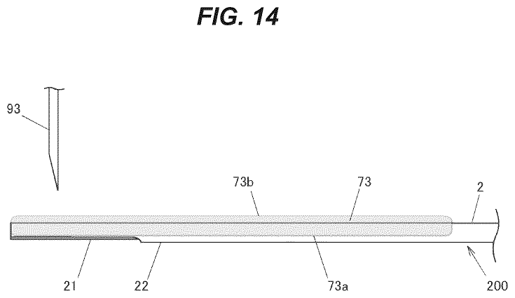

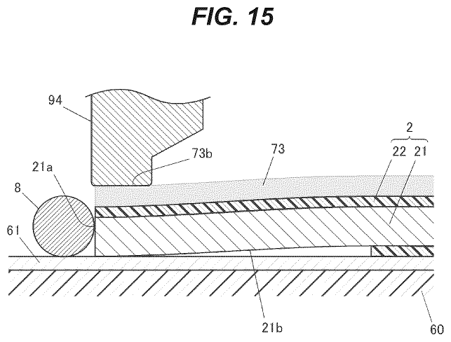

FIG. 14 is an explanatory diagram showing a specific example of the cutting step. In the cutting step, while the plurality of insulated electric wires 2 are integrated by the electric wire fixing member 73, the plurality of insulated electric wires 2 together are cut together with the electric wire fixing member 73 by the cutting tool 93. In the example shown in FIG. 14, the plurality of insulated electric wires 2 and electric wire fixing members 73 are cut along a direction perpendicular to the longitudinal direction of the plurality of insulated electric wires 2, but as in the modified example described with reference to FIGS. 6A and 6B, the plurality of insulated electric wires 2 and the electric wire fixing members 73 may be cut along a direction inclined to the longitudinal direction of the plurality of insulated electric wires 2. As in the modified example described with reference to FIGS. 7A to 8B, the end surface 21a of the core wire 21 of each of the plurality of insulated electric wires 2 may be an inclined plane inclined with respect to a direction perpendicular to the substrate 6. The cutting step may be performed after the removal step or before the removal step.

FIG. 15 is an explanatory diagram showing a specific example of the connection step. In the connection step, at least a part of the peripheral surface 21b of the core wire 21 exposed in the removal step is connected to the pad 61 together with the end surface 21a of the core wire 21. The length in the longitudinal direction of the insulated electric wire 2 of the portion of the peripheral surface 21b of the core wire 21 exposed is, e.g., 0.5 mm or more.

In the connection step, the pressing jig 94 is used to solder the tips of the plurality of insulated electric wires 2 from which a portion of the circumferential direction of the insulation coating 22 has been removed, while pressing the tips toward the pad 61. The pressing jig 94 contacts the upper surface 73b of the electric wire fixing member 73 and presses the plurality of insulated electric wires 2 through the electric wire fixing member 73 by pressing the tip of the plurality of insulated electric wires 2 by the pressing jig 94, the core wire 21 of the portion from which a circumferential portion of the insulation coating 22 is removed is bent so as to approach the pad 61.

Soldering of the core wire 21 to the pad 61 may be done by heating and melting a spherical solder ball 80 placed on the connection surface 61a of the pad 61, as shown in FIG. 15. It is not limited to this, but may be done by heating and melting the cream solder applied to the pad 61, or by pick-up soldering on the end surface 21a of the core wire 21 of the insulated electric wire 2, or by spraying molten solder.

This second embodiment also makes it possible to easily connect the plurality of insulated electric wires 2 to the substrate 6 while suppressing the occurrence of short circuits, as in the first embodiment. According to the second embodiment, the plurality of insulated electric wires 2 are arranged in parallel at intervals, which widens the spacing of pads 61 and makes it possible to more reliably prevent short circuits between the pads 61, and even when the diameter of the insulated electric wires 2 is formed larger than the standard dimension, the position of the core wire 21 of each of the multiple insulated electric wires 2 can be aligned with the position of the pad 61. In addition, since the core wire 21 is covered by the insulation coating 22 except for a part of the circumferential direction at the tip of the plurality of insulated electric wires 2, it is possible to prevent the core wire 21 from being bent, as in the comparative example shown in FIG. 5, for example. Further, since the core wire 21 in the portion from which the insulation coating 22 is removed is bent so as to approach the pad 61, the distance between the end surface 21a of the core wire 21 and the pad 61 is shortened and the core wire 21 can be more securely joined to the pad 61.

Modified Example of the Second Embodiment

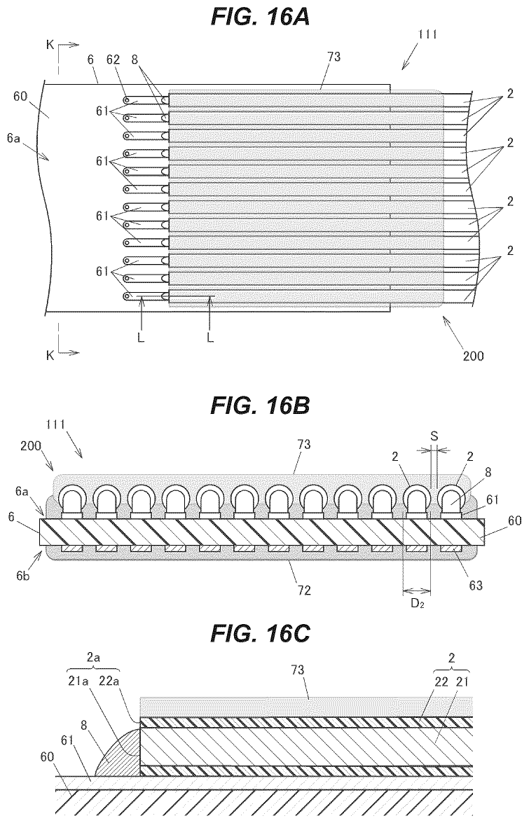

Next, the electric wire connection 111 according to a modified example of the second embodiment will be described with reference to FIGS. 16A and 16B. FIG. 16A is a configuration diagram of the electric wire connection 111 pertaining to the modified example, viewed from one side 6a of the substrate 6. FIG. 16B is a K-K line cross-sectional view of FIG. 16A, and FIG. 16C is an L-L line cross-sectional view of FIG. 16A.

The electric wire connection 111 for this modified example is manufactured without the removal step of the second embodiment, and a circumferential portion of the insulation coating 22 is not removed at the tip of the plurality of insulated electric wires 2. The other configuration is the same as in the second embodiment.

This modified example also makes it possible to easily connect the plurality of insulated electric wires 2 to the substrate 6 while suppressing the occurrence of short circuits and preventing short circuits between the pads 61, and even when the diameter of the insulated electric wires 2 is formed larger than the standard dimension, the position of the core wire 21 of each of the multiple insulated electric wires 2 can be aligned with the position of pad 61.

Summary of the Embodiments and the Modified Examples

Next, the technical concepts that can be grasped from the first and second embodiments and modified examples described above will be described with the help of the characters, etc. in these embodiments and modified examples. However, each character in the following description is not limited to the members, etc. specifically shown in the embodiments in the scope of claims.

According to the first feature, an electric wire connection structure is provided, in which a core wire 21 is connected to a plurality of pads 61 provided on a substrate 6 by an electrically conductive bonding material (solder) 8 at the tip in the longitudinal direction of each of a plurality of insulated electric wires 2, where the outer periphery of the core wire 21 is covered by an insulation coating 22, wherein, for each of the electric wires, an end surface 21a of the core wire 21 and an end surface 22a of the insulation coating 22 are at the same position at the tip in the longitudinal direction, and the electrically conductive bonding material 8 adheres to the end surface 21a of the core wire 21 and the pads 61.

According to the second feature, in the electric wire connection structure as described in the first feature, a peripheral surface 21b of the core wire 21 at each tip of the plurality of insulated electric wires 2 is covered by the insulation coating 22.

According to the third feature, in the electric wire connection structure as described in the first feature, a circumferential portion of the insulation coating 22 at the tip of each of the plurality of insulated electric wires 2 is removed to expose a peripheral surface 21b of the core wire 21, and the electrically conductive bonding material 8 adheres to the exposed peripheral surface 21b of the core wire 21.

According to the fourth feature, in the electric wire connection structure as described in the third feature, each of the plurality of insulated electric wires 2 is bent in such a manner that the core wire 21 at the tip approaches the pad 61.

According to the fifth feature, in the electric wire connection structure as described in the first feature, the plurality of insulated electric wires 2 are arranged parallel to each other on the substrate 6 and the end surfaces 21a of the core wire 21 of each are aligned linearly.

According to the sixth feature, in the electric wire connection structure as described in the fifth feature, the positions of the end surfaces 21a of the core wires 21 of the plurality of insulated electric wires 2 on the substrate 6 are aligned in a straight line inclined to the longitudinal direction of the plurality of insulated electric wires 2.

According to the seventh feature, in the electric wire connection structure as described in the first feature, the end surface 21a of the core wire 21 of each of the plurality of insulated electric wires 2 is an inclined plane inclined with respect to a direction perpendicular to the substrate 6, and the inclined plane is oriented toward the pad 61.

According to the eighths feature, in the electric wire connection structure as described in the first feature, the end surface 21a of the core wire 21 of each of the plurality of insulated electric wires 2 is an inclined plane inclined with respect to a direction perpendicular to the substrate 6, and the inclined plane is oriented toward the opposite side to the pad 61.

According to the ninth feature, in the electric wire connection structure as described in the first feature, the plurality of insulated electric wires 2 are integrated on the substrate 6.

According to the tenth feature, in the electric wire connection structure as described in the ninth feature, the plurality of insulated electric wires 2 are spaced apart from each other and arranged in parallel.

According to the eleventh feature, in the electric wire connection structure as described in the first feature, the diameter D₂₁ of the core wire 21 is 0.10 mm or less.

According to the twelfth feature, an electric wire connection method for connecting a plurality of insulated electric wires 2, each of which has the outer periphery of a core wire 21 covered by an insulation coating 22, to a plurality of pads 61 provided on a substrate 6 at their respective longitudinal tips includes an alignment step of arranging the plurality of insulated electric wires 2 parallel to each other along a predetermined alignment direction, a cutting step of trimming each of the plurality of insulated electric wires 2 so that the end surface 21a of the core wire 21 and the end surface 22a of the insulation coating 22 are at the same position in the longitudinal direction at the tip, and a connecting step of placing the plurality of insulated electric wires 2 on the substrate 6 and connecting the end surface 21a of the core wire 21 to each of the plurality of pads 61.

According to the thirteenth feature, the electric wire connection method as described in the twelfth feature, further includes a removal step of removing a circumferential portion of the insulation coating 22 at the tip of the plurality of insulated electric wires 2 to expose a peripheral surface 21b of the core wire 21, wherein in the connecting step, at least a part of the peripheral surface 21b of the core wire 21 is connected to the pad 61 together with the end surface 21a of the core wire 21.

According to the fourteenth feature, the electric wire connection method as described in the twelfth feature, further includes an integrating step of integrating the plurality of insulated electric wires 2 arranged in the aligning step, wherein the cutting step trims the core wire 21 and the insulation coating 22 with the plurality of insulated electric wires 2 in an integrated state.

According to the fifteenth feature, in the electric wire connection method as described in the fourteenth feature, the plurality of insulated electric wires 2 are spaced apart from each other in the aligning step.

According to the sixteenth feature, in the electric wire connection method as described in the fourteenth feature, in the cutting step, one end of the plurality of insulated electric wires 2 is trimmed so that the position of the end surface 21a of the core wire 21 of each of the plurality of insulated electric wires 2 on the substrate 6 is aligned in a straight line inclined to the longitudinal direction of the plurality of insulated electric wires 2.

According to the seventeenth feature, in the electric wire connection method as described in the twelfth feature, in the cutting step, the end surface 21a of the core wire 21 of each of the plurality of insulated electric wires 2 is made an inclined plane inclined with respect to the longitudinal direction, and in the connecting step, the end surface 21a of the core wire 21 is connected to the pad 61 by placing the plurality of insulated electric wires 2 on the substrate 6 so that the end surface 21a of the core wire 21 is oriented toward the pad 61.

According to the eighteenth feature, in the electric wire connection method as described in the twelfth feature, the end surface 21a of the core wire 21 of each of the plurality of insulated electric wires 2 is made inclined with respect to the longitudinal direction in the cutting step, and in the connecting step, the plurality of insulated electric wires 2 are placed on the substrate 6 so that the end surface 21a of the core wire 21 is oriented opposite the pad 61, and the end surface 21a of the core wire 21 is connected to the pad 61.

According to the nineteenth feature, a medical device (multi-electrode catheter) 1 with a catheter cable 10 having a plurality of insulated electric wires 2, the core wires 21 of which are covered by an insulation coating 22, and one of the two ends of which is inserted into the human body in the longitudinal direction, and a substrate 6 having a plurality of pads 61 to which the core wires 21 of the plurality of insulated electric wires 2 are connected, wherein the core wire 21 of each of the plurality of insulated electric wires 2 and the plurality of pads 61 of the substrate 6 are connected by the electric wire connection structure described in any one of the first to eleventh features.

According to the twentieth feature, a method for manufacturing a medical device 1 with a catheter cable 10 having a plurality of insulated electric wires 2, the core wires 21 of which are covered by an insulation coating 22 and one of the two ends in the longitudinal direction is inserted into the human body, and a substrate 6 having a plurality of pads 61 to which the core wires 21 of the plurality of insulated electric wires 2 are connected, wherein the core wire 21 of each of the plurality of insulated electric wires 2 and the plurality of pads 61 are connected by the electric wire connection method described in any one of the twelfth to eighteenth features.

The first and second embodiments of the invention and modified examples thereof have been described above, but these embodiments and modified examples do not limit the invention as claimed. It should also be noted that not all of the combinations of features described in the embodiments are essential for the invention to solve the problems of the invention.

In the first and second embodiments and modified examples thereof described above, the case in which the invention is applied to a multi-electrode catheter 1, which is a type of medical device, is described, but the invention is not limited to this case; for example, the invention may be applied to an endoscope, or the invention may be applied to a device other than a medical device.

1. An electric wire connection structure, comprising:
a plurality of insulated electric wires each with an outer periphery of a core wire being covered by an insulation coating, the core wire being connected by an electrically conductive bonding material to each of a plurality of pads provided on a substrate at a tip in a longitudinal direction of each of the plurality of insulated electric wires,
wherein, for each of the plurality of insulated electric wires, an end surface of the core wire and an end surface of the insulation coating are at a same position at the tip in the longitudinal direction, and the electrically conductive bonding material adheres to the end surface of the core wire and the pads.

2. The electric wire connection structure, according to claim 1, wherein a peripheral surface of the core wire at each tip of the plurality of insulated electric wires is covered by the insulation coating.

3. The electric wire connection structure, according to claim 1, wherein a circumferential portion of the insulation coating at the tip of each of the plurality of insulated electric wires is removed to expose a peripheral surface of the core wire, and the electrically conductive bonding material adheres to the exposed peripheral surface of the core wire.

4. The electric wire connection structure, according to claim 3, wherein each of the plurality of insulated electric wires is bent in such a manner that the core wire at the tip approaches the pad.

5. The electric wire connection structure, according to claim 1, wherein the plurality of insulated electric wires are arranged parallel to each other on the substrate and the end surfaces of respective core wires are aligned linearly.

6. The electric wire connection structure, according to claim 5, wherein positions of the end surfaces of the respective core wires of the plurality of insulated electric wires on the substrate are aligned in a straight line inclined to the longitudinal direction of the plurality of insulated electric wires.

7. The electric wire connection structure, according to claim 1, wherein the end surface of the core wire of each of the plurality of insulated electric wires is an inclined plane inclined with respect to a direction perpendicular to the substrate, and the inclined plane is oriented toward the pad.

8. The electric wire connection structure, according to claim 1, wherein the end surface of the core wire of each of the plurality of insulated electric wires is an inclined plane inclined with respect to a direction perpendicular to the substrate, and the inclined plane is oriented toward an opposite side to the pad.

9. The electric wire connection structure, according to claim 1, wherein the plurality of insulated electric wires are integrated on the substrate.

10. The electric wire connection structure, according to claim 9, wherein the plurality of insulated electric wires are spaced apart from each other and arranged in parallel.

11. The electric wire connection structure, according to claim 1, wherein a diameter of the core wire is 0.10 mm or less.

12. An electric wire connection method for connecting each of a plurality of insulated electric wires each with an outer periphery of a core wire being covered by an insulation coating to each of a plurality of pads provided on a substrate at a tip in a longitudinal direction of each of the plurality of insulated electric wires, the method comprising:
an alignment step of arranging the plurality of insulated electric wires parallel to each other along a predetermined alignment direction;
a cutting step of trimming each of the insulated electric wires in such a manner that an end surface of the core wire and an end surface of the insulation coating are at a same position at the tip in the longitudinal direction; and
a connecting step of placing the plurality of insulated electric wires on the substrate and connecting the end surface of the core wire to each of the plurality of pads.

13. The electric wire connection method, according to claim 12, further comprising:
a removal step of removing a circumferential portion of the insulation coating at the tip of each of the plurality of insulated electric wires to expose a peripheral surface of the core wire, wherein in the connection step, at least a part of the peripheral surface of the core wire exposed in the removal process is connected to the pad together with the end surface of the core wire.

14. The electric wire connection method, according to claim 12, further comprising:
an integrating step of integrating the plurality of insulated electric wires arranged in the aligning step, wherein the cutting step trims the core wire and the insulation coating with the plurality of insulated electric wires in an integrated state.

15. The electric wire connection method, according to claim 14, wherein the plurality of insulated electric wires are spaced apart from each other in the aligning step.

16. The electric wire connection method, according to claim 14, wherein in the cutting step, one ends of the plurality of insulated electric wires are trimmed and aligned in such a manner that a position of the end surface of the core wire of each of the plurality of insulated electric wires on the substrate is aligned in a straight line inclined to the longitudinal direction of the plurality of insulated electric wires.

17. The electric wire connection method, according to claim 12, wherein in the cutting step, the end surface of the core wire of each of the plurality of insulated electric wires is made an inclined plane inclined with respect to the longitudinal direction, and in the connecting step, the plurality of insulated electric wires are arranged on the substrate in such a manner that the end surface of the core wire is oriented toward the pad and the end surfaces of the core wire are connected to the pads.

18. The electric wire connection method, according to claim 12, wherein in the cutting step, the end surface of the core wire of each of the plurality of insulated electric wires is made an inclined plane inclined with respect to the longitudinal direction, and in the connecting step, the plurality of insulated electric wires are arranged on the substrate in such a manner that the end surface of the core wire is oriented toward an opposite side to the pad and the end surfaces of the core wire are connected to the pads.

19. A medical device, comprising:
a catheter cable comprising the plurality of insulated electric wires each with the core wire being covered by the insulation coating; and
the substrate comprising the plurality of pads to which the core wires of the plurality of insulated electric wires are connected,
wherein one of two longitudinal end portions of the catheter cable is configured to be inserted into a human body,
wherein the respective core wires of the plurality of insulated electric wires and the plurality of pads of the substrate are connected by the electric wire connection structure according to claim 1.

20. A method for manufacturing a medical device provided with a catheter cable comprising the plurality of insulated electric wires each with the core wire being covered by the insulation coating, and the substrate comprising the plurality of pads to which the core wires of the plurality of insulated electric wires are connected, wherein one of two longitudinal end portions of the catheter cable is configured to be inserted into a human body, the method comprising:
connecting the respective core wires of the plurality of insulated electric wires to the plurality of pads of the substrate by the electric wire connection method according to claim 12.
